(19) United States
(12) Patent Application Publication
(10) Pub. No.: US 2024/0090895 A1
(43) Pub. Date: Mar. 21, 2024

(54) FREE NEEDLE WITH SUTURE FIXATION MECHANISM

(71) Applicant: Biomet Manufacturing, LLC, Warsaw, IN (US)

(72) Inventor: Preston McCurdy, Fort Wayne, IN (US)

(21) Appl. No.: 18/367,743

(22) Filed: Sep. 13, 2023

Related U.S. Application Data
(60) Provisional application No. 63/407,260, filed on Sep. 16, 2022.

(51) Int. Cl. A61B 17/06; A61B 17/04

(52) U.S. Cl. CPC A61B 17/06004; A61B 17/0485

(57) ABSTRACT

Various systems, methods and apparatuses are shown and described including a system for tissue repair. The system optionally includes: a needle, a kite and securement mechanism. The kite can be configured to couple with the needle and forms a loop. The loop is configured to receive one or more sutures. The securement mechanism can be configured to be moveable relative to the loop of the kite to selectively engage the one or more sutures such that the one or more sutures are captured between the loop and the securement mechanism.

### CLAIM OF PRIORITY

This application claims the benefit of U.S. Provisional Patent Application Ser. No. 63/407,260, filed on Sep. 16, 2022, the benefit of priority of which is claimed hereby, and which is incorporated by reference herein in its entirety.

### FIELD

The present disclosure relates to systems, devices and methods for surgical tissue repair, and more particularly to systems, devices and methods for coupling suture to a needle without the use of a knot.

### BACKGROUND

In the human body, tissue can require repair. Such tissue includes bone, muscles, tendons, ligaments and cartilage. Forceful twisting, trauma or rotation of the knee, shoulder (or other joint) can tear or otherwise damage tissue. Disease can also necessitate replacement of bone(s) of the joint with one or more prosthetic components. Such replacement can require repair of soft tissue during the surgery. Thus, a surgical repair of the tissue may be required in various circumstances. Such repair can include suturing. Various suture assemblies have been developed for facilitating suturing and are effective for their intended purposes. Nevertheless, tissue repair assemblies and systems for facilitating suturing are still desirable.

### SUMMARY

The present disclosure provides apparatuses, methods and systems including suture(s), kite, needle and/or a securement mechanism. Such apparatuses, systems and methods that can be used to repair tissue, stabilize a joint including by suturing soft tissue or can be used for other surgical purposes for which a suture and needle are utilized.

The present inventor has realized that certain aspects of soft tissue repair procedures including those during an arthroplasty procedure can be overly complex and time consuming. In particular, attaching one or more sutures to a needle can be overly complex and time consuming. Although surgical apparatuses such as eyelets, loops, anchors and other instruments have been developed, many of these features require knots or other manipulation of the suture by the surgeon to facilitate securement of the suture to the instrument in question. Needles generally utilize the eyelet to which the suture is affixed by a knot. While tying a single knot may not seem overly complex or time consuming, needles may be coupled to and decoupled from multiple sutures at a time. Tying a knot for each of the multiple sutures can become time consuming and complex.

The present inventor has recognized an assembly with a kite configured to capture one or more sutures with the aid of a securement mechanism. The one or more sutures can be free to pass through a loop formed by the kite. The securement mechanism (e.g., a button, telescoping component, etc.) can be attached to the kite and can be moveable relative to the kite to reduce a size of the loop holding the one or more sutures and thereby capture the suture between the reduced loop of the kite and the securement mechanism. This configuration can save time and can reduce surgical complexity as multiple sutures can be captured and knots need not be utilized to attach the suture to the needle. Reduced time and complexity can result in lower surgical costs among other benefits.

The above discussion is intended to provide an overview of subject matter of the present patent application. It is not intended to provide an exclusive or exhaustive explanation of the invention. The description below is included to provide further information about the present patent application. Further areas of applicability will become apparent from the description provided herein. The description and specific examples in this summary are intended for purposes of illustration only and are not intended to limit the scope of the present disclosure.

To better illustrate the apparatuses, systems and methods disclosed herein, a non-limiting list of examples is provided here:

Example 1 is a system for tissue repair, the system optionally including: a needle; a kite configured to couple with the needle and forming a loop, wherein the loop is configured to receive one or more sutures; and a securement mechanism configured to be moveable relative to the loop of the kite to selectively engage the one or more sutures such that the one or more sutures are captured between the loop and the securement mechanism.

Example 2 is the system of Example 1, wherein the securement mechanism includes a plurality of passages therein configured to receive parts of the kite and allow for selective movement of the securement mechanism relative to the kite.

Example 3 is the system of any one or combination of Examples 1-2, wherein the securement mechanism includes an interior cavity in communication with the plurality of passages.

Example 4 is the system of any one or combination of Examples 1-3, wherein the securement mechanism forms a surface at an end of the interior cavity adjacent the plurality of passages, and wherein the surface is abutted by the one or more sutures when the one or more sutures are captured between the loop and the securement mechanism.

Example 5 is the system of any one or combination of Examples 1-4, wherein the securement mechanism is configured as one of a button or a telescoping component.

Example 6 is the system of any one or combination of Examples 1-5, wherein the telescoping component is moveable from a first position over the needle to a second position along the loop of the kite.

Example 7 is the system of any one or combination of Examples 1-6, wherein the securement mechanism is configured to be moveable along the kite to reduce a size of a portion of the loop that receives the one or more sutures prior to capture of the one or more sutures between the loop and the securement mechanism.

Example 8 is an assembly for tissue repair optionally including: a needle; a kite coupled to the needle at a base and having a capture portion extending from the base that forms a loop, wherein the loop is configured to receive one or more sutures; and a securement mechanism coupled to the kite and moveable relative to the loop of the kite to selectively engage the one or more sutures such that the one or more sutures are captured between the loop and the securement mechanism.

Example 9 is the assembly of Example 8, wherein the securement mechanism includes a plurality of passages therein configured to receive the capture portion of the kite and allow for selective movement of the securement mechanism relative to the kite along the capture portion.

Example 10 is the assembly of any one or combination of Examples 8-9, wherein the securement mechanism forms a surface at an end of an interior cavity adjacent the plurality of passages, and wherein the surface is abutted by the one or more sutures when the one or more sutures are captured between the loop and the securement mechanism.

Example 11 is the assembly of any one or combination of Examples 8-10, wherein the securement mechanism is configured as one of a button or a telescoping component.

Example 12 is the system of any one or combination of Examples 8-11, wherein the telescoping component is moveable from a first position over the needle to a second position along the loop of the kite.

Example 13 is a system for tissue repair, the system optionally including: a needle having an eyelet; and a kite forming a loop that is selectively insertable through the eyelet of the needle, wherein the loop is configured to receive one or more sutures; wherein the kite is selectively moveable to draw the loop and the one or more sutures received in the loop through the eyelet.

Example 14 is the system of Example 13, wherein upon receiving the one or more sutures in the eyelet, the needle is configured to be crimped to capture the one or more sutures within the eyelet.

Example 15 is a method of attaching one or more sutures to a needle for repair of tissue, the method optionally including: providing the needle with a kite attached thereto; passing the one or more sutures through a loop of the kite; and moving a securement mechanism relative to the loop to selectively engage the one or more sutures such that the one or more sutures are captured between the loop and the securement mechanism.

Example 16 is the method of Example 15, wherein providing the needle the kite includes attaching the kite to a distal end of the needle.

Example 17 is the method of any one or combination of Examples 15-16, wherein moving the securement mechanism relative to the loop includes reducing a size of a portion of the loop that receives the one or more sutures prior to capture of the one or more sutures between the loop and the securement mechanism.

Example 18 is the method of any one or combination of Examples 15-17, wherein moving the securement mechanism relative to the loop to selectively engage the one or more sutures includes passing the one or more sutures into an interior cavity of the securement mechanism and abutted the one or more sutures against an interior surface that forms a part of the interior cavity.

Example 19 is the method of any one or combination of Examples 15-18, wherein moving the securement mechanism relative to the loop includes telescoping the securement mechanism from the needle.

Example 20 is the method of any one or combination of Examples 15-19, further including moving the securement mechanism in an opposing direction along the loop back toward the needle to selectively disengage the securement mechanism from the one or more sutures.

Example 21 is any one or combination of the apparatus, systems and method examples above including any one or combination of the features disclosed herein.

### DRAWINGS

The above-mentioned and other features and advantages of this disclosure, and the manner of attaining them, will become more apparent and the disclosure itself will be better understood by reference to the following description of examples taken in conjunction with the accompanying drawings, wherein:

FIG. 1 is a perspective view of a system and an assembly and illustrates a suture passed through a loop of a kite coupled to a distal end of a needle, according to an example of the present application.

FIG. 1A is a cross-sectional view of a button utilized with the system and assembly of FIG. 1, according to an example of the present application.

FIG. 2 is a cross-sectional view of the button of FIG. 1A used with the kite and in the process of capturing the suture, according to an example of the present application.

FIG. 3 is a perspective view of the system and assembly of FIG. 1 with the suture now coupled to the needle via the kite and button, according to an example of the present application.

FIG. 3A is a cross-sectional view of the button used with the kite and securing the suture for use with the needle, according to an example of the present application.

FIG. 4A is perspective view of a system and an assembly and illustrates a suture passed through a loop of a kite coupled to a distal end of a needle, according to an example of the present application.

FIG. 4B is a perspective view of the system and assembly of FIG. 4A with the suture now coupled to the needle via the kite and a securement mechanism, according to an example of the present application.

FIG. 5A is perspective view of a system and an assembly and illustrates a suture passed through a loop of a kite and the kite passed through an eyelet of a needle, according to an example of the present application.

FIG. 5B is perspective view of a system and an assembly and illustrates a suture passed through the eyelet of the needle as facilitated by the kite, according to an example of the present application.

FIG. 5C is perspective view of a system and an assembly and illustrates a suture captured by crimping the eyelet of the needle, according to an example of the present application.

FIG. 6 is a flow chart of a method that can be used for tissue repair, according to an example of the present application.

### DETAILED DESCRIPTION

In describing the examples of the disclosure illustrated and to be described with respect to the drawings, specific terminology will be used for the sake of clarity. However, the disclosure is not intended to be limited to any specific terms or illustrations used herein, and it is to be understood that each specific term includes all technical equivalents.

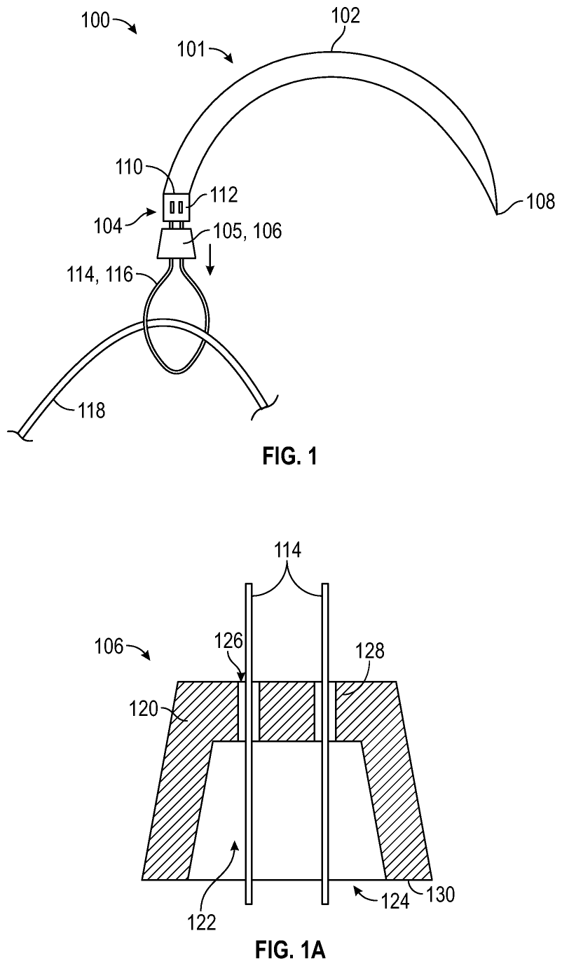

FIG. 1 illustrates a system 100 that can include a needle 102, a kite 104 and a securement mechanism 105 such as a button 106. The needle 102, the kite 104 and the button 106 can be coupled together as an assembly 101.

The needle 102 can be any type of surgical needle as known in the art including a bone cutting needle, for example. Thus, the needle can be pointed, spatulated, reverse cutting, tapercut, straight cutting, conventional cutting, quill barbed, etc. Although illustrated as curved in the example of FIG. 1, the needle 102 can be straight or have another shape (quarter circle, half circle, ⅜ circle, ⅝ circle, j-shape, compound curved, etc.). The proximal end 108 of the needle 102 can have a sharp. A distal end 110 can be coupled to the kite 104. Although the kite 104 is illustrated as a separated component that is coupled to the needle 102 by swaging, crimping, soldering, welding or other mechanical fastening technique, it is contemplated the needle 102 and kite 104 can be constructed as a single component according to other examples. Thus, the terms needle and kite need not be construed as connoting separate components.

The kite 104 can include a base 112 configured to couple to the distal end 110 of the needle 102. The base 112 can be at a proximal portion of the kite 104 and can be configured to anchor the remainder of the kite 104 to the needle 102. The kite 104 can include a capture portion 114 configured to form a loop 116 at a distal portion thereof. The capture portion 114 can be constructed of wire (e.g., nitinol wire, stainless steel wire, etc.), suture (nylon, polypropylene, etc.), band, or other suitable construct that is able to form the loop 116 and has sufficient strength.

FIG. 1 shows one or more sutures 118 received by and passing through the loop 116 of the kite 104. The one or more sutures 118 can of any type and number. Thus, sutures of different sizes, types, colors and shapes are contemplated. Any type of suture as known in the art, (e.g., broadband, ribbon, round, mesh, braided, monofilament, metal, polymer, etc.) can be utilized. The system 100 can utilize multi-loaded or single-loaded suture.

The button 106 can be configured to couple with the kite 104, in particular the capture portion 114. The button 106 can be selectively moveable along the capture portion 114 to reduce as size of the loop 116 holding the one or more sutures 118 as further illustrated and described herein.

FIG. 1A is a cross-section of an example of the button 106. The button 106 can be constructed of any suitable biologically stable material such as metal alloy, rigid plastic, composite or the like. The button 106 can comprise a shell 120 being at least partially hollow and having an interior cavity 122 with an opening 124. The shell 120 can also form a plurality of passages 126 to receive the capture portion 114 of the kite 104 and allow the capture portion 114 to pass into the interior cavity 122.

The button 106 can have a proximal end 128, where the plurality of passages 126 are located and a distal end 130 forming the opening 124. The button 106 is illustrated as having a taper from the proximal end 128 to the distal end 130. However, further examples of the button 106 can be untampered, can have a reverse tapered or can have another construction. The proximal end 128 can be configured to engage/abut with the base 112 (FIG. 1) of the kite 104. However, it is contemplated that the button 106 can be shaped with dedicated features such as tabs, hooks, recess, etc. to facilitate locking of the button 106 to the base 112 or needle 102 (FIG. 1). The shell 120 could also be constructed of non-rigid (e.g., flexible and/or deformable) material so as to be collapsible according to some examples. The moveable walls of a non-rigid shell 120 can be used to capture the capture portion 114 and/or suture, for example.

The plurality of passages 126 allow for sliding movement of the button 106 along the capture portion 114 of the kite 104. According to further examples the button 106 could have interior or distal features such as moveable locking tabs or crimping mechanisms for selective locking engagement with the capture portion 114 to affix the button 106 in a set position on the kite 104.

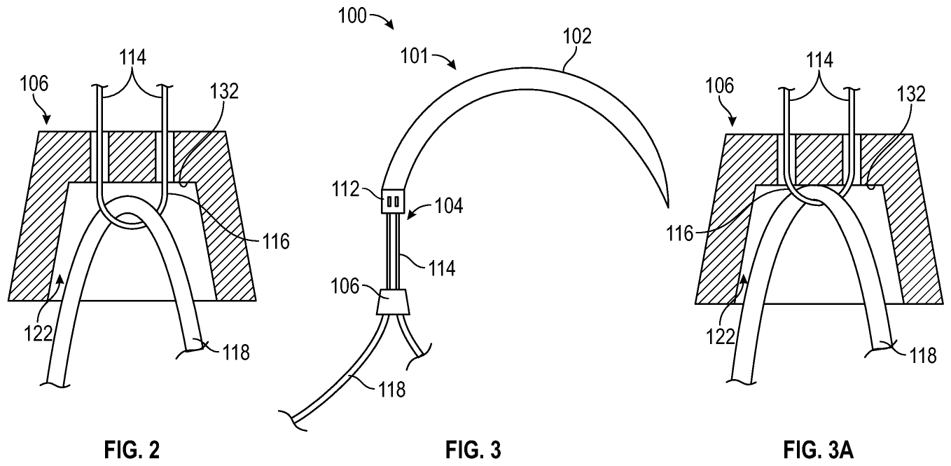

FIG. 2 shows the process of the button 106 being moved along the capture portion 114 to pull the capture portion 114 tight. Movement of the button 106 toward a distal end of the capture portion 114 reduces a size of the loop 116 remaining distal of the button 106 (i.e., this action reduces the size of the loop 116 in which the one or more sutures 118 pass). The movement of the button 106 can cause the size of the distal part of the loop 116 distal of the button 106 to shrink such that the one or more sutures 118 eventually enter into the interior cavity 122 and are brought in a position toward an interior capture surface 132 that forms an end of the interior cavity 122.

FIG. 3 shows the system 100 constructed as the assembly 101 with the button 106 capturing the one or more sutures 118 via the capture portion 114. Thus, the one or more sutures 118 are coupled to the needle 102. The needle 102 can be manipulated to pass through soft tissue or bone with the one or more sutures 118 locked to the needle 102 by the button 106 and kite 104. In this locked arrangement the one or more sutures 118 are able to follow the path of the needle 102 through the tissue. Once the assembly 101 and captured one or more sutures 118 are passed through the tissue in a desired manner, the process described above can be reversed by moving the button 106 proximally back toward the base 112 of the kite 104. This movement of the button 106 can release the one or more sutures 118.

FIG. 3A shows locking engagement of the one or more sutures 118 by the interior capture surface 132. In particular, the loop 116 is fully reduced pulling the capture portion 114 carrying the one or more sutures 118 into a locked position against the interior capture surface 132 of the button 106.

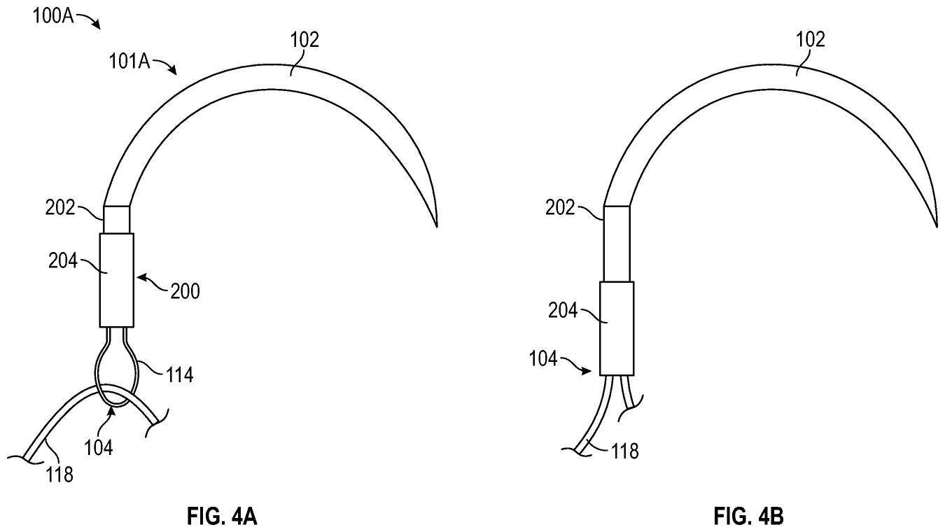

FIG. 4A shows another example of a system 100A and an assembly 101A configured to capture the one or more sutures 118. The system 100A and assembly 101A can be constructed in a manner similar to that of system 100 and assembly 101 described previously. The system 100A and assembly 101A include the needle 102 and the kite 104 as previously described. The system 100A and assembly 101A differ in that rather than the button as the securement mechanism a different construction for a securement mechanism 200 is utilized. The securement mechanism 200 includes a needle extension portion 202 and a telescoping component 204.

The needle extension portion 202 can be swaged, soldered, welded, formed as part of or otherwise coupled to the distal end 110 of the needle 102. The telescoping component 204 can be movable over the needle extension portion 202 distally down along the capture portion 114 of the kite 104 to engage and capture the one or more sutures 118 in a manner similar to that of the button as previously described and illustrated. Thus, the telescoping component 204 can have a construction similar to that of the button as previously described save that the telescoping component 204 can also selectively engage with and/or move over the needle extension portion 202 in the position of FIG. 4A. FIG. 4B shows the process of the telescoping component 204 being slid or otherwise moved off the needle extension portion 202 of the needle 102 and down along the capture portion (not shown in FIG. 4B) of the kite 104. This action moves the telescoping component 204 against the one or more sutures 118 to lock the one or more sutures 118 to the needle 102.

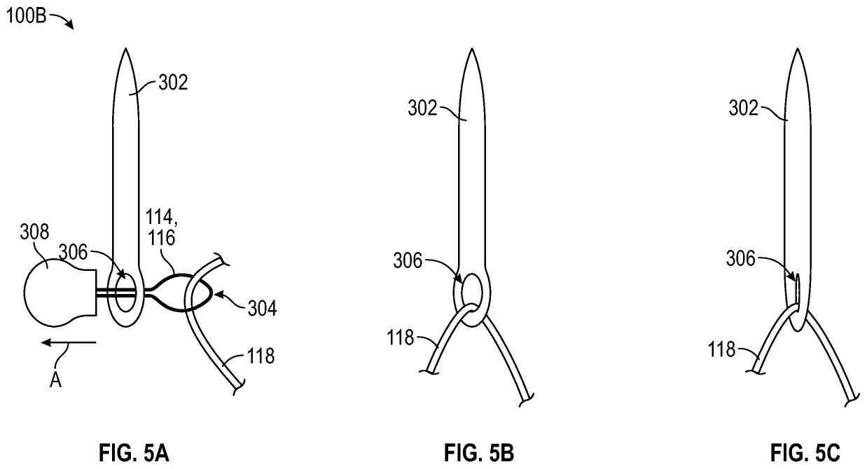

FIG. 5A shows another example of a system 100B. The system differs from those described previously. In particular, the system 100B includes a needle 302 and a kite 304.

The needle 302 is illustrated as a straight needle in FIG. 5A. However, the needle 302 can be curved or otherwise shaped in a manner such as those previously described previously with regard to the needle 102. The needle 302 includes an eyelet 306 therein. The kite 304 can include a handle 308 and the capture portion 114 forming a loop 116 as previously described.

As shown in FIG. 5A, the capture portion 114 can be inserted through the eyelet 306 such that the handle 308 of the kite 304 is on an opposing side of the needle 302 from the loop 116. The one or more sutures 118 can be passed through the loop 116.

As shown by the arrow A in FIG. 5A, the kite 304 can be moved to draw the capture portion 114 back through the eyelet 306. This can draw the one or more sutures 118 through the eyelet 306 as shown in FIG. 5B when the movement is completed. The kite 304 can be removed from engagement with the one or more sutures 118 leaving the one or more sutures 118 passing through the eyelet 306.

FIG. 5C contemplates closure of the eyelet 306 to lock the one or more sutures 118 to the needle 302. This can be performed by crimping the needle 302 to close the eyelet 306. However, other examples contemplate a dedicated component placed in the eyelet 306, and anchoring component utilized, one or more knots or other techniques utilized to lock the one or more sutures 118 to the needle 302.

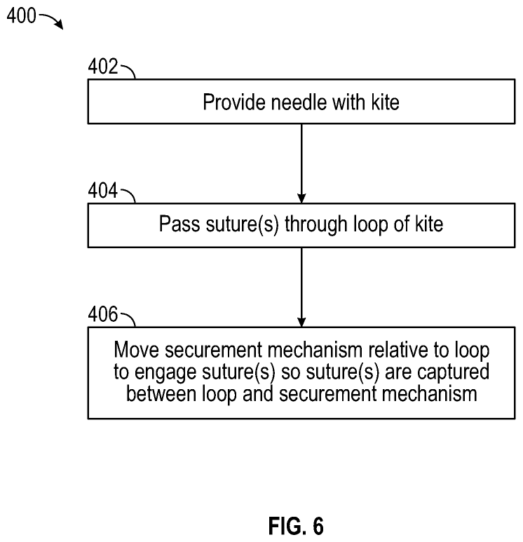

FIG. 6 is a flow chart of a surgical technique of a method 400 of attaching one or more sutures to a needle. The method 400 can include providing the needle with a kite attached thereto at step 402. The method 400 can include passing the one or more sutures through a loop of the kite at step 404. The method 400 can include moving a securement mechanism relative to the loop to selectively engage the one or more sutures such that the one or more sutures are captured between the loop and the securement mechanism at step 406. The method 400 can additionally and optionally include the kite is attached the kite to a distal end of the needle. Moving the securement mechanism relative to the loop can reduce a size of a portion of the loop that receives the one or more sutures prior to capture of the one or more sutures between the loop and the securement mechanism. Moving the securement mechanism relative to the loop to selectively engage the one or more sutures can pass the one or more sutures into an interior cavity of the securement mechanism and abutted the one or more sutures against an interior surface that forms a part of the interior cavity. Moving the securement mechanism relative to the loop can telescope the securement mechanism from the needle. The method 400 can further include moving the securement mechanism in an opposing direction along the loop back toward the needle to selectively disengage the securement mechanism from the one or more sutures.

It will be readily understood to those skilled in the art that various other changes in the details, material, and arrangements of the parts and method stages which have been described and illustrated in order to explain the nature of the inventive subject matter can be made without departing from the principles and scope of the inventive subject matter as expressed in the subjoined claims. For example, the order of method steps or stages can be altered from that described above, as would be appreciated by a person of skill in the art.

It will also be appreciated that the various dependent claims, examples, and the features set forth therein can be combined in different ways than presented above and/or in the initial claims. For instance, any feature(s) from the above examples can be shared with others of the described examples, and/or a feature(s) from a particular dependent claim may be shared with another dependent or independent claim, in combinations that would be understood by a person of skill in the art.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A system for tissue repair, the system comprising:
a needle;
a kite configured to couple with the needle and forming a loop, wherein the loop is configured to receive one or more sutures; and
a securement mechanism configured to be moveable relative to the loop of the kite to selectively engage the one or more sutures such that the one or more sutures are captured between the loop and the securement mechanism.

2. The system of claim 1, wherein the securement mechanism includes a plurality of passages therein configured to receive parts of the kite and allow for selective movement of the securement mechanism relative to the kite.

3. The system of claim 2, wherein the securement mechanism includes an interior cavity in communication with the plurality of passages.

4. The system of claim 3, wherein the securement mechanism forms a surface at an end of the interior cavity adjacent the plurality of passages, and wherein the surface is abutted by the one or more sutures when the one or more sutures are captured between the loop and the securement mechanism.

5. The system of claim 1, wherein the securement mechanism is configured as one of a button or a telescoping component.

6. The system of claim 5, wherein the telescoping component is moveable from a first position over the needle to a second position along the loop of the kite.

7. The system of claim 1, wherein the securement mechanism is configured to be moveable along the kite to reduce a size of a portion of the loop that receives the one or more sutures prior to capture of the one or more sutures between the loop and the securement mechanism.

8. An assembly for tissue repair comprising:
a needle;
a kite coupled to the needle at a base and having a capture portion extending from the base that forms a loop, wherein the loop is configured to receive one or more sutures; and
a securement mechanism coupled to the kite and moveable relative to the loop of the kite to selectively engage the one or more sutures such that the one or more sutures are captured between the loop and the securement mechanism.

9. The assembly of claim 8, wherein the securement mechanism includes a plurality of passages therein configured to receive the capture portion of the kite and allow for selective movement of the securement mechanism relative to the kite along the capture portion.

10. The assembly of claim 9, wherein the securement mechanism forms a surface at an end of an interior cavity adjacent the plurality of passages, and wherein the surface is abutted by the one or more sutures when the one or more sutures are captured between the loop and the securement mechanism.

11. The assembly of claim 8, wherein the securement mechanism is configured as one of a button or a telescoping component.

12. The assembly of claim 11, wherein the telescoping component is moveable from a first position over the needle to a second position along the loop of the kite.

13. A system for tissue repair, the system comprising:
a needle having an eyelet; and
a kite forming a loop that is selectively insertable through the eyelet of the needle, wherein the loop is configured to receive one or more sutures;
wherein the kite is selectively moveable to draw the loop and the one or more sutures received in the loop through the eyelet.

14. The system of claim 13, wherein upon receiving the one or more sutures in the eyelet, the needle is configured to be crimped to capture the one or more sutures within the eyelet.

15. A method of attaching one or more sutures to a needle for repair of tissue, the method comprising:
providing the needle with a kite attached thereto;
passing the one or more sutures through a loop of the kite; and
moving a securement mechanism relative to the loop to selectively engage the one or more sutures such that the one or more sutures are captured between the loop and the securement mechanism.

16. The method of claim 15, wherein providing the needle the kite includes attaching the kite to a distal end of the needle.

17. The method of claim 15, wherein moving the securement mechanism relative to the loop includes reducing a size of a portion of the loop that receives the one or more sutures prior to capture of the one or more sutures between the loop and the securement mechanism.

18. The method of claim 15, wherein moving the securement mechanism relative to the loop to selectively engage the one or more sutures includes passing the one or more sutures into an interior cavity of the securement mechanism and abutted the one or more sutures against an interior surface that forms a part of the interior cavity.

19. The method of claim 15, wherein moving the securement mechanism relative to the loop includes telescoping the securement mechanism from the needle.

20. The method of claim 15, further comprising moving the securement mechanism in an opposing direction along the loop back toward the needle to selectively disengage the securement mechanism from the one or more sutures.
